(19) United States
(12) Patent Application Publication
(10) Pub. No.: US 2015/0038459 A1
(43) Pub. Date: Feb. 5, 2015

(54) PROTEIN CONTAINING FOOD STUFFS

(71) Applicant: Jump Brands, LLC, Basalt, CO (US)

(72) Inventors: Michelle Bacarella, Gilbert, AZ (US); Steven A. Rittmanic, Chandler, AZ (US); Thomas D. Ingolia, Morton, IL (US)

(21) Appl. No.: 14/520,156

(22) Filed: Oct. 21, 2014

Related U.S. Application Data
(63) Continuation of application No. 13/203,222, filed on Aug. 24, 2011.
(60) Provisional application No. 61/208,355, filed on Feb. 24, 2009.
(60) Provisional application No. 61/269,137, filed on Jun. 22, 2009.

(51) Int. Cl. A23J 3/22; A61K 31/716; A23L 1/09; A23J 3/08; A23L 1/064

(52) U.S. Cl. CPC A23J 3/22; A23J 3/08; A23L 1/064; A23L 1/095; A61K 31/716; A23V 2002/00
USPC 514/54; 426/573; 426/311; 426/578

(57) ABSTRACT

Nutritious protein-containing foodstuffs can be prepared in the form of gels, including protein gel and fruit purees that contain all essential amino acids. A protein gel for use as a foodstuff is prepared by heating an aqueous protein solution to between 160 and 185 degrees Fahrenheit at an acidic pH between 2.85 and 3.5, the protein solution having a viscosity greater than 100 centipoise and containing a protein at a concentration of 10-20% w/w, the protein being at least one of a whey protein isolate and a liquid soy protein isolate, the protein gel having a viscosity of between 10,000 and 1,000,000 centipoise.

### RELATED APPLICATIONS

This application is a continuation of U.S. patent application Ser. No. 13/203,222, filed Aug. 24, 2011 and titled PROTEIN CONTAINING FOOD STUFFS, which application is a United States National Stage application of International Application No. PCT/US2010/025210, filed Feb. 24, 2010 and claiming priority from U.S. Provisional Application Nos. 61/208,355, filed Feb. 24, 2009, and 61/269,137, filed Jun. 22, 2009, all of which are incorporated herein by reference.

### BACKGROUND OF THE INVENTION

Protein is a key required nutrient. For a healthy diet, it is preferable to consume significant amounts of high quality protein at least at each meal. Requirements for protein are increased in children, the aged, and in athletes or others recovering from injury or during and/or after extended physical activity.

Whey and soy proteins are examples of high quality protein sources for human nutrition, with ideal amino acid composition, taste, and digestibility. Other abundant proteins, such as collagen, are inferior in their amino acid composition and completely lacking in required amino acids. Supplementing these protein sources with the missing nutritive ingredients is expensive and often unpalatable.

Small amounts of protein between meals are believed to be effective in curbing appetite and have advantages over carbohydrate-based snacks. For this purpose, it is desirable to have a stable, palatable, and easily portable configuration of the high quality protein-containing food. Currently there are two main types of such portable protein-containing food preparations: those with low water content such as bars and jerky, and those with high water content such as liquid protein drinks.

Dried meats, such as jerky, are an example of a low-water content protein source with good stability and portability. Protein bars and cakes are another example of a low-water content source of portable, stable high quality protein. Texture, taste and other aspects of jerkys and protein bars limit the scope of their appeal. Liquid protein drinks represent another configuration in which high quality protein can be provided. With protein drinks, portability and stability are relatively limited. There remains a need in the art for protein foodstuffs with significant levels of high quality protein in intermediate water levels, such as might be provided in the form of squeezable gels and fruit purees.

Gels have a wide variety of applications in food products, ranging from peripheral applications, such as meat extenders, to direct use in squeezable food products. Squeezable products are particularly useful, because they can provide important nutrients for soldiers, athletes, children at school, malnourished individuals, and others for whom transportability, time, and convenience are important factors.

Syrups and squeezable gels are used by consumers today; examples include Clif Shot® gel, GU Roctane Ultra Endurance Energy® gel, Powerbar® gel, and Powerbar® gel Blasts®. Gels are typically available in 1 to 2 ounce single-serve packs, particularly for use among athletes. However, these products are composed principally of carbohydrates and contain little or no protein. There is a need for protein during extended physical activity, such as marathon running, bicycling, and combat, but current sports gels address this need by incorporating amino acids in amounts less than 500 mg per serving, which is inadequate nutritionally and causes undesirable bitterness. There are also protein-containing gel preparations utilizing collagen and similar types of structural or connective tissue protein as the protein source. These proteins form gels easily but are deficient or completely lacking in required amino acids, and must be supplemented with other proteins, protein hydrolyzates, and/or amino acids to make them suitable for supporting human nutrition. The supplementation process is expensive and often leads to bitter, unpalatable products. A familiar example is gelatin, which is typically used to form a gel by dissolving a gelatin-containing powder in boiling water and cooling the dissolved mixture; the gel forms after cooling.

One hindrance to the preparation and marketing of gels containing nutritious protein is that upon heating the concentrated protein solutions to temperatures necessary to facilitate microbially stable products, the protein solution gels to a hardness that is inappropriate for incorporation into packages, making manufacturing difficult or impossible.

Thus, there remains an unmet need to have a manufacturable food preparation in gel or other form containing high levels of high quality protein. The present invention meets this and other needs.

### SUMMARY OF THE INVENTION

The present invention provides protein foodstuff containing nutritious and/or high quality protein in stable, palatable, easily transportable, intermediate water-content preparations and methods for making such foodstuffs. This invention also provides methods for combining nutritious and/or high quality protein with other foods and food supplements in a convenient and cost-effective manner, and protein formulations useful in those methods and foodstuffs produced by those methods.

In a first aspect, the present invention provides methods for making the protein gels of the invention. These methods involve use of aqueous protein sources, heating, acidification prior to heating, the incorporation of agents in addition to the protein solutions to facilitate appropriate viscosities after heating, and incorporation of other useful agents. In various embodiments, agents that facilitate appropriate gelling in the manufacture of the gels are employed, because in the absence of these agents, the extent of gelling is difficult to control, thereby preventing scale up for food production. Suitable agents include, but are not limited to, sugar syrup solutions (e.g., brown rice syrup, tapioca syrup, cane sugar syrup), fiber (soluble or insoluble), maltodextrins, galactodextrins, gums, pectins and other thickening agents.

In a second aspect, the present invention provides protein-containing gels and purees suitable for use as foodstuffs. The foodstuffs of the invention are formed from aqueous protein concentrates, including but not limited to soy protein, whey protein isolate, liquid egg white protein, partially hydrolyzed aqueous protein concentrates, and combinations thereof, all of which have excellent protein nutritional content. The protein-containing gels of the invention have excellent taste, texture and mouthfeel. The protein-containing purees of the invention include, but are not limited to, applesauce, pear, peach, banana, apricot, mango, and plum fruit purees.

These and other aspects and embodiments of the invention are described in detail below.

### DETAILED DESCRIPTION OF THE EMBODIMENTS

The presently preferred embodiments of the present invention will be best understood by reference to the examples, discussed below. It will be readily understood that the components of the present invention, as generally described in the examples, could be arranged and designed to provide a variety of different formulas and compositions. Thus, the following more detailed description and examples are not intended to limit the scope of the invention as claimed, but are merely representative of presently preferred embodiments of the invention.

This detailed description of the invention is divided into sections and subsections for the convenience of the reader. After first providing definitions (Section I) to assist the reader, the methods for making protein-containing gel foodstuffs and protein-containing puree foodstuffs of the invention are described (Section II). Then, the protein gels and protein purees and their utility are described (Section III), followed by examples illustrating how to make various illustrative foodstuffs of the invention.

### I. DEFINITIONS

“Essential amino acids” as used herein means phenylalanine, valine, threonine, tryptophan, isoleucine, methionine, leucine, cysteine, tyrosine, histidine, and arginine.

“Gel” as used herein means a protein foodstuff with a viscosity greater than 1,000 centipoise (approximate viscosity of castor oil at 20 degrees Centigrade) and less than 1,000,000 centipoise (approximate viscosity of smooth peanut butter at 25 degrees Centigrade), and a protein foodstuff with a viscosity between 10,000 centipoise (approximate viscosity of chocolate syrup at 25 degrees Centigrade) and 1,000,000 centipoise.

“High quality protein” as used herein means a protein foodstuff containing all of the essential amino acids, and with at least 15 grams of branched chain amino acids (leucine, isoleucine, and/or valine) per 100 grams protein.

“Nutritious protein” as used herein means a protein foodstuff containing all of the essential amino acids.

“Protein” as used herein means a molecule containing a polymer of at least 30 contiguous amino acids linked via amide chemical bonds.

“Protein foodstuff” as used herein means a food suitable for human consumption containing at least 2% w/w protein.

“Protein gel” as used herein means a gel containing nutritious and/or high quality protein.

“Protein puree” as used herein means a puree containing nutritious and/or high quality protein.

“Puree” as used herein means a blended and/or homogenized foodstuff. Purees are often made from fruit, and have a viscosity greater than water but less than smooth peanut butter.

### II. METHODS FOR PREPARING PROTEIN GELS AND PUREES

This section describes methods of the invention for creating protein gels and protein purees and their key ingredients and composition. First, key process parameters and the importance of utilizing certain protein ingredients, pasteurizing and gelling temperatures, rate of temperature increase, and acidification, are discussed. Then, the importance of viscosity, and the use of syrups, fibers, and other viscous agents, in the production of protein gels with manageable and useful viscosities is discussed. Finally, the flexibility or amendability of the methods of the invention, which allow incorporation of other useful ingredients, is described.

Generally, however, the present invention provides, in one aspect, a method for making protein-containing gels and purees suitable for use as foodstuffs. The foodstuffs of the invention are formed from aqueous protein concentrates, including but not limited to soy protein, whey protein isolate, liquid egg white protein, partially hydrolyzed aqueous protein concentrates, and combinations thereof, all of which have excellent protein nutritional content. The protein-containing gels of the invention have excellent taste, texture and mouthfeel. The protein-containing fruit purees of the invention of the invention include, but are not limited to, applesauce, pear, peach, banana, apricot, mango, and plum fruit purees.

Thus, in a first aspect, the invention provides a method of forming a gel for use as a foodstuff, said method comprising heating an aqueous protein solution at an acidic pH and sufficient viscosity to induce gelling, wherein said protein solution is heated to a temperature of at least about 100 degrees Fahrenheit until said protein gel forms. This method involves various key elements, including use of aqueous protein; heating to effect gelling; acidification prior to heating; use of facilitating agents to help control extent of gelling; and incorporation of other useful ingredients, each of which is discussed in the subsections below. In the following subsection, the use of nutritious and/or high quality proteins in aqueous form, and the advantages of using this form of starting material versus powdered or other forms of protein in the methods of the invention are described.

a. Aqueous Protein

The protein foodstuffs of the invention are formed from aqueous protein concentrates, including but not limited to soy protein, whey protein isolate, and partially hydrolyzed aqueous protein concentrates. These sources of protein are nutritious proteins, containing all essential amino acids, and usually are high quality proteins, typically containing at least 15 grams of branched chain amino acids per 100 grams of protein. These protein sources also have excellent taste, texture, and mouthfeel.

Use of aqueous concentrated protein allows preparation of a wide variety of gels and purees containing combinations of ingredients for a variety of applications and nutritional profiles. Use of aqueous concentrated protein allows preparation of gels without air incorporation, thereby facilitating production of gels with the desired mouthfeel and without grittiness. At concentrations of protein above about 5% (w/w), no gelling agent beyond protein need be added to provide the desired viscosity and consistency, particularly when combined with agents contributing viscosity to the starting ingredients before heating. At lower levels of protein, fruit purees can be used to increase the viscosity of the protein foodstuff. Air incorporation and foaming are problems associated with use of powdered protein because of the high amount of mixing energy needed to re-solubilize the protein; use of aqueous protein sources overcomes these problems. Finished product benefits include elimination of undesirable gritty mouthfeel, and higher protein levels attainable. The methods of the invention also lead to a lower inherent microbial load, especially yeast and mold, which can be associated with the spray drying process for proteins.

Some embodiments of the invention utilize aqueous protein at a starting protein concentration of between 20 and 30% w/w. Other embodiments utilize aqueous protein at a starting protein concentration of between 1 and 50% w/w. Still further, other embodiments utilize protein at a concentration of between 15 and 20% w/w, between 1 and 20% w/w, between 1 and 10% w/w, between 10 and 20% w/w, and greater than 10% w/w.

Some embodiments of the present invention utilize an aqueous protein solution having a viscosity in the range of 100 to 1000 centipoise. Other embodiments utilize a protein solution having a viscosity of less than 10,000 centipoise. Still further, other embodiments utilize a protein solution having a viscosity greater than 100 centipoise. The aqueous protein is combined with other liquid ingredients and an appropriate amount of water in a suitable vessel with light to moderate stirring, taking care to avoid air incorporation.

This mixture, which typically initially has a pH of between 6 and 7, is acidified to about pH 3.3 by addition of acid. In one embodiment, 85% orthophosphoric acid is used as acidulant. In other embodiments, other food compatible acids are used. Other ingredients are then added sequentially or as a pre-blend of dry ingredients, with the final addition of organic or other acid added to bring the pH to the desired acidic pH. In one embodiment, the pH is 3.1. In another embodiment, the initial pH adjustment is to between 2.8 and 3.9, with the final pH also adjusted to between 2.8 and 3.9. Still further, in some embodiments the initial pH adjustment is to between 2.85 and 3.5.

The mixture is heated to both pasteurize and cause the desired gelling. This may be done by mixing with slow heating in a steam-jacketed vessel, or it may be accomplished in a more continuous fashion by pumping the material through a tubular heat exchanger, both of which methods are commonly used for processing viscous solutions. In one embodiment, the mixture (protein solution) is heated from room temperature to 120 degrees Fahrenheit with stirring at a rate of about 1 degree per minute. In another embodiment, the solution is heated from between 30 degrees Fahrenheit and 110 degrees Fahrenheit to between 115 and 200 degrees Fahrenheit. In another embodiment, the heating is carried out at a rate of between 0.1 and 100 degrees per minute. The time and temperature parameters needed for this step vary depending upon the protein percentage in the product, the relative amounts of other solutes, the desired amount of gelation, and the pasteurization requirements.

Once gelation has occurred, the resulting gel is then typically packaged for distribution and use. In one embodiment of the invention, gel is extruded into Seal-A-Meal® bags used for sealing foods for storage, and the bags are heat-sealed. In another embodiment, the gel is extruded into typical form-fill-seal methods utilizing, for example, multilayer aluminum plastic material that is commonly used for this type of food.

Use of aqueous protein concentrates has several processing, cost, and finished-product advantages over use of dry protein ingredients. Use of aqueous protein concentrates eliminates several processing problems associated with use of dry protein ingredients, and has advantages related to process, product range possibilities, and protein levels in the finished product.

The invention provides a number of other benefits and advantages. The invention provides a simple and low cost way to incorporate large amounts of protein into gels and/or fruit purees. Large amounts of protein can be incorporated into a stable, palatable, portable food, providing benefits with respect to nutrition and convenience to the consumer. Products are shelf stable for at least a year, have appropriately low microbial levels, excellent taste and mouthfeel, and the viscosity can be adjusted to that desired by the customer. Manufacturing methods are rapid, with no need for long incubation times or time-intensive processing. Formulations for the protein foodstuffs described in this invention are flexible, allowing a wide variety of flavors and colorants to be added with a low risk of inactivating or damaging the additives. Use of the invention allows management of the viscosity of the final product after heating, thereby facilitating incorporation into packaging for marketing. Importantly, the invention provides methods for reducing inappropriate over-gelling during heating of the product through utilization of agents contributing viscosity, an effect counter-intuitive and counter to currently held beliefs by those skilled in the art.

In one embodiment, the aqueous protein concentrate used is liquid whey protein isolate. This product is available from Trega Foods, Wisconsin USA, and is usually obtained with a protein concentration of between 27% and 31% w/w. In another embodiment, other aqueous protein sources are used including soy protein, egg white protein, and protein hydrolyzates.

The aqueous protein sources discussed above, including whey protein isolate, soy protein isolate, and egg white protein, are distinguished from non-nutritious sources of protein, such as gelatin and other structural and/or connective tissue proteins, in ways other than their nutritional value. For example, the way the proteins react to heating and the way they form gels are different. Gelatin and similar non-nutritious proteins are typically used to form a gel by heating in boiling water until dissolved and then allowing the solution to cool in a mold. If this process is used with nutritious proteins such as whey protein isolate and soy protein isolate, an irreversible process leads to formation of an insoluble mass during the heating process. It is therefore critical to manage the heating process as a function of the ingredients and their concentration in the starting solution. The next section describes the heating process in more detail.

b. Heating

In the methods of the invention, the aqueous protein concentrates are typically mixed with other ingredients, acidified, and heated to at least 100 degrees Fahrenheit and up to 210 degrees Fahrenheit while being stirred to effect gelling. The gelling of the protein solution occurs during the heating, and the extent and rate of gelling depends on temperature, rate of temperature increase, protein concentration, presence of other ingredients, and other factors. Other factors being equal, higher pasteurization temperatures lead to higher resulting viscosities. In one embodiment of the invention, the final pasteurization temperature is between 120 and 210 degrees Fahrenheit. In a further embodiment of the invention, the final pasteurization temperature is between 160 and 185 degrees Fahrenheit. Finally, in some embodiments the final pasteurization temperature is between 160 and 200 degrees Fahrenheit. In certain embodiments, gelling of protein solutions of 15% w/w and higher is initiated at temperatures as low as 120 degrees Fahrenheit, and viscosities greater than 100,000 centipoise (100,000 centipoise is approximately the viscosity of corn syrup) are reached at temperatures as low as 150 degrees Fahrenheit. At lower protein concentrations, the extent of gelling is less, viscosities reached are less, and the temperature at which the gelling commences is higher.

The higher the rate of temperature increase during the heating step, the more rapid the initiation of gelling and the higher the final viscosity reached. In one embodiment, the rate of temperature increase is between 0.1 and 100 degrees Fahrenheit per minute. In one embodiment, with a rate of temperature increase at the higher end of this range and a final pasteurization temperature of 190 degrees Fahrenheit, a 16.7% w/w protein solution reached a viscosity greater than 1,000,000 centipoise (1,000,000 centipoise is the approximate viscosity of smooth peanut butter). In another embodiment, the rate of temperature increase is between 5 and 30 degrees Fahrenheit per minute. In yet another embodiment, the rate of temperature increase is between 6 and 15 degrees Fahrenheit per minute.

As discussed above, the protein-containing solutions are typically acidified prior to the heating step. The acidification method is described in the next subsection.

c. Acidification

Acidification of the protein solution is important for several reasons. Those skilled in the art will appreciate that acidified foods, particularly those with a pH below 4.0, are more stable and resistant to microbial degradation than non-acidic foods. Acidified foods are also subject to lesser regulatory scrutiny. In addition, acidification of protein solutions can change the conformation of the constituent proteins and affect the gels that are formed. In this invention, the protein solutions, if not prepared at an acidic pH, are acidified prior to the heating and concomitant gel formation.

Acidification of the protein-containing solution prior to heating and gelling is typically required for the protein solutions useful in the methods of the invention. Acidification can be carried out with any strong acid that is safe for foodstuffs, including but not limited to orthophosphoric acid, hydrochloric acid, citric acid, malic acid, and tartaric acid. In one embodiment, the protein solution is acidified at room temperature to a pH of between 2.8 and 3.9 before heating. In another embodiment, the protein solution is acidified to a pH of between 3.0 and 3.5 before heating. Still further, in other embodiments the protein solution is either acidified to a pH of between 2.85 and 3.5, or between 2.85 and 4.1 before heating. In yet another embodiment, the protein solution can be further acidified after heating.

Acidification followed by heating will lead to gelling of the concentrated protein solutions described and exemplified herein. The next subsection describes methods and agents to control gel formation; counterintuitively, the agents that reduce viscosity of the final gel are themselves significantly more viscous than water.

d. Agents to Control the Gelling Process

In various embodiments of the invention, one or more agents that provide manageable viscosities (also referred to herein as “facilitating agents”) during and after heating are employed. In the absence of incorporation of these agents, the extent of gelling can be difficult to control, preventing cost-effective scale up of the method. For example, over-gelling can occur with some proteins at some concentrations without use of these agents, leading to an overly solid mass that cannot be filled into pouches. Prior to the present invention, those skilled in the art might think that over-gelling could be avoided by reducing the viscosity of the starting preparation, such as by using highly potent artificial sweeteners instead syrups. However, such approaches do not work with the protein solutions of the invention; instead, the problem can be avoided by the addition of agents such as sugar syrup solutions (e.g., brown rice syrup, tapioca syrup, cane sugar syrup), fiber (soluble or insoluble), maltodextrins, galactodextrins, gums, pectins, and other thickening agents. Although the present invention is not to be bound by any theory of mechanism of action, a common feature of these agents is that they increase the viscosity of the starting solution.

In one embodiment, sugar syrup solutions are employed in the methods of the invention. One particular embodiment is agave syrup, which may be added to the protein solution to provide from 10-50% w/w syrup in the protein solution. For example, 38% w/w agave syrup is shown in Example 1 below. In another embodiment, the agave syrup is used at between 1 and 60% w/w. Specifically, in some embodiments the agave syrup is used at a concentration between 25-50% w/w, 5-10% w/w, and 15-20% w/w. In other embodiments, the sugar syrup is brown rice syrup, tapioca syrup, or cane sugar syrup.

In other embodiments, the facilitating agent used is a fiber. In one embodiment, VitaSugar™ iso-malto-oligosaccharide from BioNeutra, a soluble fiber, at about 1 to 40% w/w, is employed. In another embodiment, the VitaSugar soluble fiber (isomalto-oligosaccharide) is used at 5-20% w/w, as described in Example 5 below. In other embodiments, the fiber facilitating agent used is another soluble fiber such as Nutriose® (Roquette) or Palatinose, or an insoluble fiber. In another embodiment, the facilitating agent is maltodextrin. In one particular embodiment, the maltodextrin is chain length 9-13, a product available from Cargill, and is used at between 1 and 60% w/w. In another embodiment, the maltodextrin is used at about 10-15% w/w, as described in Example 6 below. In other embodiments, the maltodextrin has an average chain length longer than 13; in yet other embodiments, the maltodextrin has an average chain length less than 9. In other embodiments, the facilitating agents are galactodextrins, gums, pectins and other thickening agents.

The foregoing components and method steps, i.e., aqueous protein, heating, acidification, and facilitating agents, can be used to produce a wide variety of useful protein gels and protein purees. While these foodstuffs are described in more detail below, it is important to appreciate that the methods of the invention are flexible and allow other useful ingredients to be incorporated into the foodstuffs of the invention. The next subsection accordingly describes supplemental ingredients that can be incorporated into the protein foodstuffs of this invention and the advantages conferred by addition of these ingredients.

e. Other Ingredients

In various embodiments of this invention, other ingredients are incorporated into the gel or puree. Particular embodiments of other ingredients include but are not limited to vitamins and vitamin mixtures, minerals, nutraceuticals, weight loss agents, and weight gain agents. These agents can be added singly or in various combinations.

One embodiment of such an ingredient is WellMune™, supplied by Biothera, Inc, an agent hypothesized to support immune function. A particular embodiment of the present invention contains Wellmune at a level of 100-500 mg per serving. Another embodiment, described in Example 4 below, contains Wellmune at 200-400 mg per serving.

Another embodiment of an additional ingredient is oil, such as canola oil at 5% w/w. A further embodiment is canola oil at between 0.1 and 25% w/w. Other embodiments include other types of oils, such as medium chain length triglyceride (MCT) oil or safflower oil. Other embodiments include sweeteners such as sucrose, stevia, sucralose, and other natural and artificial sweeteners. Further embodiments are flavorants, colorants, and similar additives; a wide variety of flavorants and colorants can be incorporated, as exemplified in Examples 1 through 6 below.

In the preceding section, the methods of the invention for production of useful protein foodstuffs have been described. In the next section, foodstuffs produced by the methods are described.

### III. PROTEIN FOODSTUFFS

The present invention provides novel protein-containing gels and purees suitable for use as foodstuffs. The foodstuffs of the invention are formed from aqueous protein concentrates, including but not limited to soy protein, whey protein isolate, liquid egg white protein, and partially hydrolyzed aqueous protein concentrates, which are composed of nutritious and high quality protein. Methods for producing these protein foodstuffs have been described above.

The protein foodstuffs themselves are described below. At high starting protein concentrations, the resulting products are protein gels, while at lower starting protein concentrations, the resulting products are protein purees. The gels and purees of the invention have excellent taste, texture and mouthfeel, and have the added advantages of being portable, stable, and nutritious. While there are other gels in the marketplace, none of the presently available gels have the concentration or amount of nutritious and/or high quality protein as those of the present invention. Gels formed from structural protein or other similar protein, such as gelatin and/or collagen, are markedly nutritionally deficient and form different types of gels formen in markedly different ways.

a. Protein Gels

In one embodiment, the foodstuff produced is a protein gel. Gels have a wide variety of applications in food products, ranging from peripheral applications, such as meat extenders, to direct use in squeezable food products. Squeezable products are particularly useful, because they can provide important nutrients for soldiers, athletes, children at school, malnourished individuals, and others for whom transportability, time, and convenience are important factors. Syrups and squeezable gels and are used by consumers today; examples include Clif Shot gel, GU Roctane Ultra Endurance Energy gel, Powerbar gel, and Powerbar gel Blasts. Gels are typically available in 1 to 2 ounce single-serve packs, particularly for use among athletes. However, these products are composed principally of carbohydrates and contain little or no protein. The present invention meets the need for protein-containing foodstuffs for these applications and others by providing a protein foodstuff that can be conveniently consumed during extended physical activity, such as marathon running, bicycling, and combat, as a portable snack, and with meals. In one embodiment, the serving size of the protein gels is between about ⅓ ounce (about 10 grams) and 5 ounces (about 150 grams). In another embodiment, the serving size is between about one ounce (about 30 grams) and three ounces (about 90 grams).

The previous section describes protein gels, which arise from the methods of the invention when high starting protein concentrations are employed. At lower starting protein concentrations, protein can be added to purees to obtain foodstuffs with desired viscosity, taste, texture, and mouthfeel. The next subsection describes protein purees.

b. Purees

In another embodiment, the invention provides foodstuffs in the form of purees. Attempts to incorporate powdered protein from a wide variety of sources into purees were unsuccessful, leading to incompletely dissolved, partially opaque and unpalatable preparations. Aqueous protein concentrates solved this problem and allowed the creation of excellent protein-fortified purees. Aqueous protein sources suitable for use in making the purees of the invention include, but are not limited to liquid whey protein isolate, liquid soy protein isolate, liquid egg white protein, and protein hydrolyzates. Purees suitable for addition of protein include, but are not limited to, applesauce and purees of pear, peach, banana, apricot, mango and plums. It is difficult to dissolve powdered proteins in these fruit purees without compromising the taste, texture and/or appearance of the purees. Use of aqueous protein concentrates causes minimal perturbation of these and other attributes, resulting in protein-enhanced fruit purees with little indication to the consumer of the presence of protein in the product. In some embodiments, a fruit puree is provided having a fruit concentration of 10-99% w/w. In other embodiments, a fruit puree is provided having a fruit concentration of 20-98% w/w.

In one embodiment, the protein is combined with applesauce to form a puree of the invention. In one particular embodiment, shown in Example 3 below, applesauce and protein are mixed, and then phosphoric acid is added slowly with mixing to bring the pH to 3.5. Other ingredients, such as flavors, colors, and vitamins, are added at this point. Final pH adjustment to 3.3 is accomplished by addition of malic acid. The mixture is then heated in a double boiler at a rate of about 1 degree Fahrenheit per minute to about 170 degrees Fahrenheit, with constant stirring, or to a temperature at which the desired amount of gelation is achieved.

In another embodiment, the solution is heated from between 30 degrees Fahrenheit and 110 degrees Fahrenheit to between 115 and 230 degrees Fahrenheit. In another embodiment, the heating is carried out at a rate of between 0.1 and 100 degrees per minute.

In any of these embodiments, the heated mixture may then be placed in a suitable container, such as a plastic cup or Seal-a-Meal bags. In another embodiment, the product solution is incorporated into typical form-fill-seal methods utilizing, for example, multilayer aluminum plastic material.

In one embodiment, the serving size of the protein applesauce is between one ounce (about 30 grams) and 10 ounces (about 300 grams). In another embodiment, the serving size is about 4 ounces (about 120 grams), which is about the amount commonly contained in cups of applesauce sold at retail outlets. Other embodiments utilize other fruit purees, including but not limited to purees of pear, peach, banana, apricot, mango and plums.

One embodiment of the invention utilizes liquid whey protein isolate as the starting protein, a product that can be purchased from Trega Foods in Wisconsin, USA. Another embodiment utilizes liquid soy protein isolate. Yet other embodiments utilize partially hydrolyzed aqueous protein concentrates.

c. Benefits of the Foodstuffs of the Invention

The foodstuffs of the invention provide a number of benefits and advantages. Consumption of the foodstuffs of the invention provides a simple and low cost source of large amounts of nutritious and/or high quality protein. The foodstuffs are stable, palatable, and portable, providing benefits with respect to nutrition and convenience to the consumer. Products are shelf stable for at least a year, have appropriately low microbial levels, excellent taste and mouthfeel, and the viscosity can be adjusted to that desired by the customer. The protein foodstuffs described can include any of a wide variety of flavors and colorants to be added, thereby enhancing the attractiveness of the food and making it easier to consume these nutritious foods instead of non-nutritious snack foods.

The following examples describe illustrative preferred embodiments of these inventions and methods of making them. It will be apparent to those skilled in the art that the examples are but one of a variety of ways to combine the ingredients and attributes desired.

### EXAMPLES

### Example 1

This example describes how to make a foodstuff of this invention that was designed for athletes and contains protein, carbohydrate and calories in a ratio preferred by student sports organizations, such as the NCAA®. This formulation combined aqueous whey protein and agave syrup as the principal ingredients. In this formulation, agave syrup contributed to the viscosity of the starting material, thereby facilitating the flowability of the final product after heating. Fruit punch flavorant and a natural red colorant were also utilized, but a wide variety of flavorants and colorants and other nutrients such as vitamins can be combined with the two principal ingredients. The following formulation (Table 1) was for one 60 g serving of fruit punch-flavored gel.

TABLE 1

| | Amount for 1 | |
|---|---|---|
| Food Item | 60 g serving | |
| Aqueous whey protein concentrate, 1-50% w/w protein | 35 | g |
| Agave nectar, Wholesome Sweeteners, Light Organic | 23 | g |
| Blue Agave | | |
| Colormaker Natural Red 0417 | 0.057 | g |
| Fruit Punch flavor, Virginia Dare, Nat Wet AN28 | 0.057 | g |
| WONF 16279 | | |
| Vitamin D and B's Premix Watson WT-11212 | 0.024 | g |
| Orthophosphoric acid, 85% | Ca. 1.1 | g * |
| Citric Acid, anhydrous | Ca. 0.28 | g ** |

| | |
|---|---|
| Water | To bring to 60 g |

* to bring pH to ca. 3.3
** to bring pH to 3.1

The aqueous whey protein and agave syrup were combined in a glass vessel with light to moderate stirring, taking care to avoid air incorporation. This mixture, which initially had a pH of between 6 and 7, was acidified to about pH 3.3 by addition of 85% orthophosphoric acid. The other ingredients were then added sequentially, however it is also possible to first pre-blend the dry ingredients, and then add the pre-blended ingredients to the mixture. The final addition was citric acid to bring the pH to the desired level of 3.1. The mixture was heated to both pasteurize and cause the desired gelling. This was done by mixing with slow heating in a pan, but can also be accomplished in a steam-jacketed vessel or it may be accomplished in a more continuous fashion by pumping the material through a tubular heat exchanger, both of which methods are commonly used for processing viscous solutions. The time and temperature parameters needed for this step vary depending upon the protein percentage in the product, the relative amounts of other solutes, the desired amount of gelation, and the pasteurization requirements. With the above formula in this example, the solution was heated from room temperature to about 165 degrees Fahrenheit with stifling at a rate of about 1 degree per minute. When the mixture thickened to the desired viscosity, it was removed from the heat and extruded into a sealable container. In the case of laboratory samples, Seal A Meal bags used for sealing foods for storage were used. Bags were heat-sealed on the bottom and sides, the material was extruded into the top, and the top was heat-sealed. In an industrial setting, product could be filled into typical form-fill-seal methods utilizing, for example, multilayer aluminum plastic material which is commonly used for this type of food. Material prepared in this way is stable for extended periods at room temperature. Each 60 gram serving provides whey protein and other nutrients in a palatable and convenient gel preparation. The amount of protein can vary from zero to about 50% (w/w) of the final product.

A flavor variant of this formulation was also prepared which substituted tea flavors for the fruit punch, as described in Table 2, below.

TABLE 2

| | Amount for 1 | |
|---|---|---|
| Food Item | 60 g serving | |
| Aqueous whey protein concentrate, 1-50% w/w protein | 35 | g |
| Agave nectar | 23 | g |
| Virginia Dare Nat Wet Green Tea Flavor, TP69 | 0.12 | g |
| Virginia Dare Nat Wet Black Tea Flavor, TQ05 | 0.12 | g |
| Virginia Dare Nat Wet Roibos Red Tea Flavor, TR55 | 0.06 | g |
| Virginia Dare Nat Wet White Tea Flavor, TQ44 | 0.06 | g |
| Vitamin D and B's Premix Watson WT-11212 | 0.024 | g |
| Orthophosphoric acid, 85% | Ca. 1.1 | g * |
| Citric Acid, anhydrous | Ca. 0.28 | g ** |

| | |
|---|---|
| Water | To bring to 60 g |

* to bring pH to ca. 3.3
** to bring pH to 3.1

### Example 2

A second foodstuff was created which utilized aqueous whey protein to prepare a gel with protein and excellent mouthfeel and taste but with few calories, thereby allowing the preparation to be used as part of weight-loss regimens. This preparation, carried out as described in Example 1 above with ingredients shown below (Table 3), produced a novel high-protein weight-loss food gel preparation.

TABLE 3

| | Amount for 1 | |
|---|---|---|
| Food Item | 60 g serving | |
| Aqueous whey protein concentrate, 1-50% w/w protein | 35 | g |
| Agave nectar | 23 | g |
| Virginia Dare Nat Wet Green Tea Flavor, TP69 | 0.12 | g |
| Virginia Dare Nat Wet Black Tea Flavor, TQ05 | 0.12 | g |
| Virginia Dare Nat Wet Roibos Red Tea Flavor, TR55 | 0.06 | g |
| Virginia Dare Nat Wet White Tea Flavor, TQ44 | 0.06 | g |
| Vitamin D and B's Premix Watson WT-11212 | 0.024 | g |
| Orthophosphoric acid, 85% | Ca. 1.1 | g * |
| Citric Acid, anhydrous | Ca. 0.28 | g ** |

| | |
|---|---|
| Water | To bring to 60 g |

* to bring pH to ca. 3.3
** to bring pH to 3.1

### Example 3

A third exemplary foodstuff of the invention utilized aqueous whey protein to prepare a gel that was then homogenized to a desired consistency and combined with applesauce, although other fruit purees can be substituted for the applesauce (see Table 4). For example, Mott's brand of applesauce has a line of products called Mott's® Plus, which incorporates additional flavors and nutritional ingredients with applesauce, such as one variety with cranberry and raspberry flavor and extra fiber and vitamin C. Whey, soy or other protein can be combined with any of these formulations to provide a more nutritious and valuable food product. In some embodiments, a food product is provided having from about 1 to 50% w/w protein.

TABLE 4

| | Amount for one | |
|---|---|---|
| Food Item | 85 g serving | |
| Aqueous whey protein concentrate, 1-30% w/w protein | 34 | g |
| Unsweetened applesauce w/+Vitc-Cnd | 49.5 | g |
| Virginia Dare Natural Liquid Apple Flavor, AU02 | 42 | mg |
| SweetLeaf dry Stevia 97 Reb A | 11 | mg |
| Orthophosphoric acid, 85% | Ca. 1.3 | g * |
| Malic acid | Ca. 85 | mg ** |

| | |
|---|---|
| Water | To bring to 85 g |

* to bring pH to ca. 3.5
** to bring pH to 3.3

Applesauce and protein were mixed, and then phosphoric acid was added slowly with mixing to bring the pH to 3.5. The other ingredients, including the flavors, colors, sweeteners, were added at this point. Final pH adjustment to 3.3 was accomplished by addition of malic acid. The mixture was then heated in a double boiler at a rate of about 1 degree Fahrenheit per minute to about 170 degrees Fahrenheit, with constant stirring. The heated mixture was then placed in Seal-a-Meal bags, although other containers such as a plastic cup or foil bag, can be used.

In an alternate method, the aqueous protein solution can be pH adjusted with phosphoric acid prior to mixing with the applesauce. In this case, the mixture may be homogenized to reduce the average particle size of the protein gel to that which is typical of applesauce.

### Example 4

A fourth exemplary foodstuff of the invention incorporates a biologically-relevant amount of a nutritional supplement. Various studies suggest that soluble beta-glucan preparations can support immune function. WellMune™, supplied by Biothera, Inc, is one such compound. For example, 250 mg of WellMune per 50 gram serving was stirred in warm agave nectar until dissolved. The clear solution was allowed to cool before mixing with the aqueous whey protein, and the preparation proceeded as in the formula of Example 1, above.

### Example 5

A fifth exemplary foodstuff of the invention incorporated brown rice syrup as an agent contributing to initial viscosity as well as to sweetness. The following formula (Table 5) was followed which utilizes fruit punch flavors, but a wide variety of flavorants and colorants can be substituted.

TABLE 5

| | Amount for 1 | |
|---|---|---|
| Food Item | 60 g serving | |
| Aqueous whey protein concentrate, 1-50% w/w protein | 35 | g |
| IMO syrup, 50% (w/w) iso-malto-oligosaccharides | 10 | g |
| Brown rice syrup, 50% (w/w) carbohydrate content | 10 | g |
| Sucrose | 3 | g |
| Colormaker Natural Red 0417 | 0.057 | g |
| Fruit Punch flavor, Virginia Dare, Nat Wet AN28 | 0.057 | g |
| WONF 16279 | Ca. 1.1 | g * |
| Orthophosphoric acid, 85% | Ca. 0.28 | g ** |
| Citric Acid, anhydrous | | |

| | |
|---|---|
| Water | To bring to 60 g |

* to bring pH to ca. 3.3
** to bring pH to 3.1

The gel product was prepared as in Example 1 above.

### Example 6

A sixth exemplary foodstuff of this invention utilized maltodextrin as an agent to contribute viscosity as well as sweetness. In this example, a 50% w/w aqueous solution of MD 01960 Maldodextrin Sweetener—Dry CUI was prepared prior to mixing with the protein. The recipe utilized, as shown in Table 6 below, contains grape flavoring, but a wide variety of flavorants and colorants can be substituted.

TABLE 6

| | Amount for 1 | |
|---|---|---|
| Food Item | 60 g serving | |
| Aqueous whey protein concentrate, 1-30% w/w protein | 34 | g |
| Organic agave syrup | 10 | g |
| MD 01960 maltodextrin syrup, 50% w/w maltodextrin | 14 | g |
| content | | |
| Tartaric Acid, | 0.312 | g |
| GNT Purple natural wet Bordeaux color | 0.045 | g |
| Grape flavor, Virginia Dare, Nat Wet CS07 | 0.066 | g |
| Stevia sweetener | 0.012 | g |
| Orthophosphoric acid, 85% | Ca. 1.1 | g * |

| | |
|---|---|
| Water | To bring to 60 g |

* to bring pH to 3.1

The gel product is prepared as in Example 1 above.

### Example 7

Packaged gels can also be prepared by delivering the mixture into pouches or other suitable containers before heating, and then heating the containers to the desired temperature for the desired time. In the preparation of Example 1 above, a sealed pouch containing the mixture was submerged in a 170 degree Fahrenheit water bath for 10 minutes, thereby achieving the desired consistency and stability.

The present invention may be embodied in other compositions and forms without departing from its methods, goals or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

1. A method of forming a protein gel for use as a foodstuff, said method comprising mixing and heating an aqueous protein solution from room temperature to between 160 and 185 degrees Fahrenheit at a constant rate from approximately 0.1 to approximately 15 degrees Fahrenheit per minute, said aqueous protein solution having an acidic pH between 2.85 and 3.5, said protein solution having a viscosity greater than 100 centipoise and containing a protein at a concentration of 10-20% w/w, said protein being at least one of a whey protein isolate and a liquid soy protein isolate, said protein gel having a viscosity of between 10,000 and 1,000,000 centipoise.

2. A method of forming a protein gel for use as a foodstuff, said method comprising mixing and heating an aqueous protein solution at an acidic pH and sufficient viscosity to induce gelling, wherein said solution is heated from room temperature to a temperature of at least about 100 degrees Fahrenheit at a constant rate from approximately 0.1 to approximately 15 degrees Fahrenheit per minute until said protein gel forms.

3. The method of claim 2, wherein said protein contains all essential amino acids.

4. The method of claim 3, wherein said protein further comprises a branched chain amino acid at levels of at least 15 grams branched chain amino acid per 100 grams protein.

5. The method of claim 2, wherein said protein solution is selected from the group consisting of whey protein isolate, liquid soy protein isolate, liquid egg white protein, and protein hydrolyzates.

6. The method of claim 2, wherein said protein solution is stirred during said heating step and is heated to a temperature of at least 120 degrees Fahrenheit but not more than 210 degrees Fahrenheit.

7. The method of claim 2, wherein said protein solution has a viscosity in the range of 100 to 1,000 centipoise.

8. The method of claim 2, wherein said protein solution has a viscosity of less than 10,000 centipoise, and said protein gel has a viscosity of less than 1,000,000 centipoise.

9. The method of claim 2, wherein said protein solution is at a pH in the range of from about 2.8 to 3.9 during said heating step.

10. The method of claim 2, wherein said protein solution is adjusted to an acidic pH prior to said heating step by addition of an acid selected from the group consisting of orthophosphoric acid, hydrochloric acid, citric acid, malic acid, and/or tartaric acid.

11. The method of claim 2, wherein said protein gel is adjusted to a more acidic pH after said heating step by addition of an acid selected from the group consisting of orthophosphoric acid, hydrochloric acid, citric acid, malic acid, and/or tartaric acid.

12. The method of claim 2, wherein said protein solution has a protein concentration of from about 1 to 50% w/w protein.

13. The method of claim 12, wherein said protein concentration is selected from the group consisting of at least 10% w/w protein and not more than 10% w/w protein.

14. The method of claim 2, wherein an ingredient in addition to protein is present in said protein solution, said ingredient being selected from the group consisting of: a whey protein at a concentration in range of 1-20% w/w, a syrup at a concentration of 5-50% w/w, a flavorant, a colorant, a fiber at a concentration of 5-20% w/w, a soluble fiber at a concentration of 5-20% w/w, a fruit puree at a concentration of 20-98% w/w, a vitamin, a mineral, a nutraceutical, a weight loss agent, a weight inducing agent, an oil, a sweetener, maltodextrin at a concentration of 10-15% w/w, and any combination thereof.

15. The method of claim 14, wherein said ingredient increases the viscosity of said protein solution.

16. The method of claim 2, wherein an ingredient in addition to protein is present in said protein solution, said ingredient being selected from the group consisting of a syrup, an oil, a soluble fiber, an insoluble fiber, a dextrin, a gum, a pectin, and mixtures of any of the foregoing ingredients.

17. The method of claim 16, wherein said protein solution contains protein at a concentration of 10-20% w/w and has a pH of between 2.85 and 3.5 and a viscosity greater than 100 centipoise, and said protein solution is heated from at least about 100 degrees Fahrenheit to a maximum temperature between 160 and 185 degrees Fahrenheit at a constant rate of 5 to 30 degrees Fahrenheit per minute with mixing, and said protein gel formed by said heating step has a viscosity of between 10,000 and 1,000,000 centipoise.

18. The method of claim 16, wherein said protein solution contains protein at a concentration of 1-10% w/w and fruit puree at a concentration of 10-99% w/w and has a pH of between 2.85 and 4.1, and said protein solution is heated to between 160 and 200 degrees Fahrenheit at a rate of 5 to 30 degrees Fahrenheit per minute.

19. The method of claim 1, wherein the method further comprises continuous mixing.

20. The method of claim 2, wherein the method further comprises continuous mixing.
